(19) United States
(12) Patent Application Publication
(10) Pub. No.: US 2005/0251679 A1
(43) Pub. Date: Nov. 10, 2005

(54) Original Transcript System: Method and System to issue, submit and view original electronic transcripts.

(75) Inventor: Prasanna Narayanan, Santa Clara, CA (US)

Correspondence Address: Prasanna Narayanan, 1901 Halford Ave, #159, Santa Clara, CA, 95051, US

(73) Assignee: Prasanna Narayanan, Santa Clara, CA (US)

(21) Appl. No.: 10/709,161

(22) Filed: Apr. 18, 2004

(51) Int. Cl. C12Q 1/68

(52) U.S. Cl. 713/170

(57) ABSTRACT

Transcript in OTS refer to any electronic file that can include a PDF, word or an image file, and is issued by an issuer. The original transcript system consists of transcript servers that register and host users. These transcript servers talk securely to each other through the transcript network server. Each OTS-ID that refers to a user can act in one of the roles of issuer, applicant or reviewer for a transcript that is defined by the user type. When a transcript is issued or submitted only the envelope for transcript information is sent. A view request causes the transcript file to be downloaded directly from the issuer. OTS provides web based access of transcripts and enables sharing of transcripts by the applicant to multiple reviewers, without the issuer having to explicitly share it to reviewers.

### BACKGROUND OF INVENTION

1. Field of the Invention

A transcript in OTS is any electronic file that includes a PDF file, word file or image file. Transcripts are issued by the issuer and are, original copies of documents maintained by the issuer, or a collection of information from various sources maintained by the issuer.

Examples: An employment verification letter is a transcript. A passport book is a collection of transcripts where each page is a transcript. A university mark sheet summary is a transcript.

some examples of processes that require transcripts are . . .

process: Apply for a loan. Transcripts: Bank statement, Verification of employment.

process: Obtain auto insurance from an insurance company. Transcripts: Driving License, Proof of vehicle ownership.

process: Apply to graduate school. Transcripts: Undergraduate transcripts, Recommendation letters.

process: Apply for visa. Transcripts: Passport, Birth certificate.

process: Enter a country. Transcripts: Passport, Visa.

OTS provides a secured way for all involved parties, to log on to the OTS system for their transcript needs. Using OTS, an issuer can issue a transcript to the applicant, an applicant can receive an issued transcript from the issuer and submit it to a reviewer. An Issuer, applicant or reviewer of a transcript can view the transcript.

When all involved parties (issuer, applicant, reviewer) for a process are on OTS, it will eliminate the need to, submit in person, snail post or e-mail, supporting transcripts to complete that process. Storage of originals in any form electronic or paper by the applicant and reviewer will also be eliminated.

As an example Company XYZ, company ABC and individual Peter are all registered on OTS, and use OTS for Peter's transcript. The process is a background check by ABC on Peter's prior employment with XYZ. The transcript is relieving letter issued by XYZ for Peter.

Peter's previous company XYZ logs in to OTS and issues the relieving letter to Peter. Peter logs in to OTS submits the received relieving letter to company ABC. ABC logs into OTS and views Peter's relieving letter, using it in the background check process.

There is no e-mail or paper based exchange involved. When ABC or Peter wants to view the transcript it is transferred from XYZ. There is no storage and maintenance of the original transcript issued by company XYZ, at company ABC or Individual Peter.

2. Description of Related Art

The process of submitting an original document is one of most cumbersome activities, be it through paper or e-mail.

Paper based transcripts: Original transcripts are lost, and needs to be issued again. Also often original transcripts cannot themselves be sent out. A notarized or attested copy of the original is sent out after submitting the original before a notary. Hence submitting these paper based transcripts can be delayed and can even end up in an application getting cancelled for lack of originals. Also original transcripts when received could be cleverly forged, and are needed to be verified for authenticity.

Digital Signature: Digital signatures, needs public keys also to be transferred besides the digitally signed document. Also a reviewer of a digital document needs to know the public key, sent by the issuer to the applicant. If the public key is lost, or the certificate is revoked, the whole process of sending transcripts by e-mail and public keys must be carried out again. Additionally once the public key is known any receiver can read a digitally signed document.

Organizing transcripts: In both the paper based and digitally signed transcripts, issuers and receivers have to individually file them based on the issuers, applicants and reviewers. They have to maintain a list to who all a transcript was issued, or from who all a transcript was received from. There is a lot of manual work involved to organize these transcripts even after the identity is confirmed.

OTS overcomes these difficulties by providing secure identity, secure content, with no maintenance required by an applicant or reviewer for transcripts.

The transcripts are issued by the issuer, and sent to the applicant. An applicant can decide to whom that transcript in turn needs to be submitted for review. The original transcripts are always requested from the issuer when attempted to be viewed by an applicant or reviewer. A reviewer can receive a transcript from the issuer only if submitted to the reviewer by the applicant.

This provides the ability for the issuer to just delete the transcript, and thus end any future views of that original transcript. The control of a transcript by the issuer need not depend on the interaction with a third party, to revoke certificates.

OTS provides a secure view for transcripts. Transcripts are always generated by the issuer and transferred from the issuer to an applicant or reviewer in a secure medium.

Issuer, identity is guaranteed on any transcript in OTS, digitally signed or not. In cases where transcripts are digitally signed, the OTS—Digital Signature combination acts as a double identity assurance of the issuer.

When a transcript is issued through OTS, the issuer knows that the recipient is the person who requested the transcript. The applicant also knows who sent the transcript. The reviewer knows who the issuer and applicant of a transcript is.

OTS eliminates storing transcripts at the applicant and reviewer area. Users can retrieve transcript information for which they have a role, by search parameters that include, time issued, time received, user, description, role, file name.

### SUMMARY OF INVENTION

OTS is a system where users log in to their respective transcript servers, for their transcript needs that communicate with each other through the transcript network server The transcript servers are distributed and must be able to reach the transcript network server through the Internet.

An issuer issues a transcript to a unique user identifier (e.g. SSN or a company web site). name An applicant receives this issued transcript from the issuer, views it and submits to a company using the unique user identifier (e.g. company web site name). A reviewer receives this transcript from the applicant.

Whenever a transcript is issued, or submitted only the envelope for transcript (EFT) is passed to the receiver. When a transcript is viewed the actual transcript is sent from the issuer to the receiver.

Original Transcript System, (OTS) is a identity secure, content secure and organized means to issue and receive original transcripts.

Transcript in OTS refers to any electronic file that can include a PDF document, word document or an image file.

A user can register as a company or an individual to the OTS system by providing an unique user id (UUID). A registered user is assigned an OTS-ID that ties the user to the registered server and is unique on the entire system. This OTS-ID acts as the address for a user on the OTS system. A user can log in to the registering server using a user name local to the registering server and mapped to the OTS-ID.

The OTS system is made of transcript servers and transcript network server. The communication between transcript servers is through the transcript network server. The transcript provider provides public/private key pairs to each server on OTS. This causes a virtual private transcript network (VPTN), which enables only authorized transcript servers to communicate with each other.

A transcript server (TS) does authentication of client systems. It also sends out envelope for transcript (EFT) to other OTS-IDs, stores EFT received from other OTS-IDs, receives transcripts from an issuer and forwards it to an authorized client to complete a view request.

The transcript network server (TNS) works with the transcript servers to complete a user registration. Upon completion the transcript network server assigns an OTS-ID unique in the system to the registered user. It also ties the registered user to the transcript server the user registered on. The transcript network server maintains a mapping between registration information and OTS-IDs. It also maintains a mapping between OTS-ID and hosting transcript server.

Companies can own their own transcript servers. Individual can use transcript servers owned by the transcript provider. A transcript server must get access codes from the TP to communicate with OTS-IDs on another transcript server.

Users talk to their transcript server using clients. A client is an application that provides the user interface to talk to the transcript server in a secured manner. This client application can be a stand alone application running on the user's desktop or a application loaded from the transcript server in a web browser.

OTS defines roles for a user on a transcript. A user registers as a Company or Individual. A company user or individual user must be able to clear a background check to complete registration and have an OTS-ID assigned by the transcript network server for that user.

Only a company user can create a transcript. A transcript is created by the company user entering the transcript server as issuer and moving the transcript to the transcript server or providing a link file on the transcript server that points to the transcript on a remote computer system accessible from the transcript server. This company becomes the issuer for the transcript. When the issuer issues this transcript to another user, the recipient user becomes the applicant. The applicant can in turn submit this transcript to a company other than the issuer, for review. This company becomes the reviewer for the transcript. An individual user can only act as an applicant. A company user can act in one of the roles of issuer, applicant or reviewer for a transcript. Any user that has roles on a transcript can view that transcript.

When a transcript is issued or submitted only the EFT information is sent.

When a transcript is viewed it always comes from the issuer. This enables the issuer to stop issuing a transcript just by deleting the transcript from the issuer path on the issuer's transcript server. If the issuer owns the whole transcript server the issuer can turn of communication to the system and disable access to all transcripts on that transcript server. A user need not rely on a third party to revoke certificates on individual transcripts.

OTS eliminates the need to e-mail transcripts or snail post transcripts. OTS eliminates the need to store original transcripts by the applicant or reviewer. This is because a transcript viewed by the applicant or reviewer using OTS is always generated from the issuer and is original, secure and role identity guaranteed.

### DETAILED DESCRIPTION

### Description: Preferred Embodiment

The OTS system contains transcript servers, and the transcript network server. Transcript servers talk to each other through the transcript network server, and are the servers that users log on to for their transcript needs.

Transcript Server (TS):

TS enables a user to register with the OTS system. TS transfers the registration information to the transcript network server (TNS). TS receives from the TNS an OTS-ID for the user. OTS-ID is the address provided by the transcript network server for a user. This address is unique throughout the system and ties the user to a transcript server and registration information. Upon receiving the OTS-ID for the user, TS stores the registration information for the user, the user's OTS-ID, creates a user name and password and provides the user name and password to the user. The user can from now on log in to the registering TS using the user name and password.

Transcript servers always talk to the transcript network server. Each communication contains a sender OTS-ID and a receiver OTS-ID.

Transcript Network Server (TNS):

When registration request is received from a transcript server for a user, TNS registers the user to OTS. The registration information received by TNS must clear a background check, performed by the transcript provider. After the background check is cleared TNS assigns a OTS-ID for a user, and ties this OTS-ID to the registration information and the registering transcript server.

TNS maintains the following information for identifying a user by the unique identifier and location of the TS hosting the user, a mapping between unique user id like SSN or company web site name and the OTS-ID, and a mapping between OTS-ID and hosting transcript server.

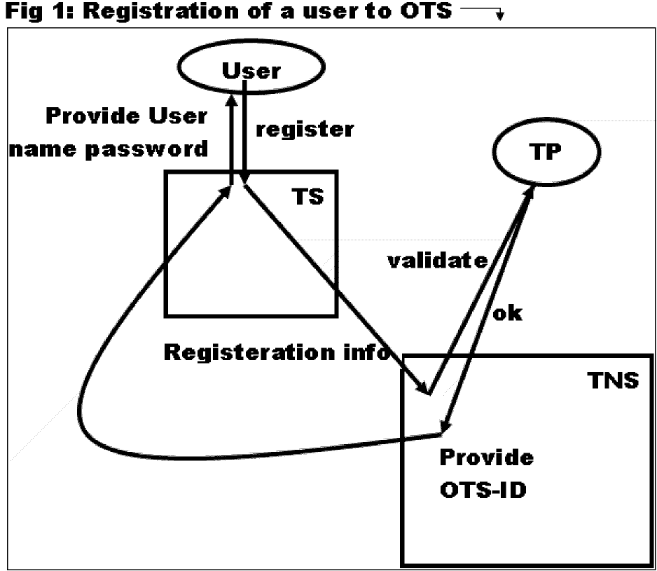

FIG. 1 shows the steps involved when a user registers to the OTS.

A transcript server communicates with other transcript servers through the transcript network server. The communication involves two phases. The first phase involves asking the transcript server the OTS-ID for a user inputted unique user id of the recipient. Once the OTS-ID is got from the TNS, the sending TS sends another message that has sender and receiver OTS-ID. The transcript network server receives this message, finds the transcript server hosting the receiver OTS-ID and forwards this message to the transcript server hosting the receiver OTS-ID.

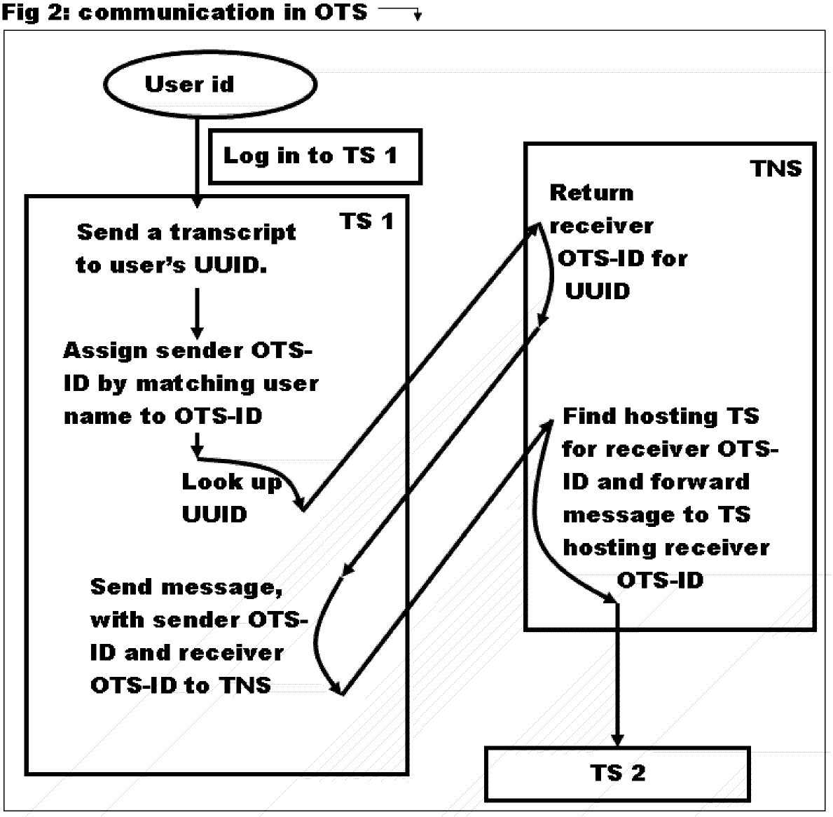

FIG. 2 shows the steps involved when two transcript servers communicate with each other through the TNS.

Transcript provider (TP):

The transcript provider is an administrative person who maintains the OTS system. The transcript provider provides private keys to each transcript server and the transcript network server. The transcript provider also provides the transcript servers with the public key of the transcript network server, and the transcript network server with the public keys of all transcript server it communicates with.

The transcript provider authenticates a user during registration and validates the registration information. Upon success, the TP directs the transcript network server to assign a OTS-ID unique on the system for the user.

Definitions:

User Types: (Company/individual):

A user on OTS is either a Company or an Individual. A company is one who owns a transcript server and logs on to that transcript server after registering to OTS with company information (e.g. EIN or web site name). An individual is one who uses a transcript server maintained by the transcript provider and logs on to that transcript server after registering to OTS with individual information (e.g. SSN or passport number).

Roles: (Issuer/Applicant/Reviewer):

Issuer, applicant and reviewer are the possible roles a user has on a transcript

Issuer: Issuers are always company users. When a company creates a transcript, it becomes the issuer of the transcript. An issuer can issue this transcript to another company user or individual user.

Applicant: Applicants are either company users or individual users. When a transcript is issued to a company or individual, the recipient user becomes the applicant. An applicant can in turn submit the issued transcript to a company user who is not the issuer.

Reviewer: Reviewers are always companies users. A company to which the applicant submits a transcript becomes the reviewer.

Action: (Issue/Submit/View)

Issue: ‘Issue’ refers to the act of an issuer issuing a transcript to an applicant.

Submit: ‘Submit’ refers to the act of an applicant submitting a transcript to a reviewer that was previously issued to the applicant.

View: All users related to a transcripts can view that transcript.

During a Issue or Submit, transcript servers do not send the actual transcript but instead send the envelope for transcript (EFT) information only.

When a view request occurs on a transcript by the issuer, applicant or reviewer the transcript is sent from the issuer path to the viewer.

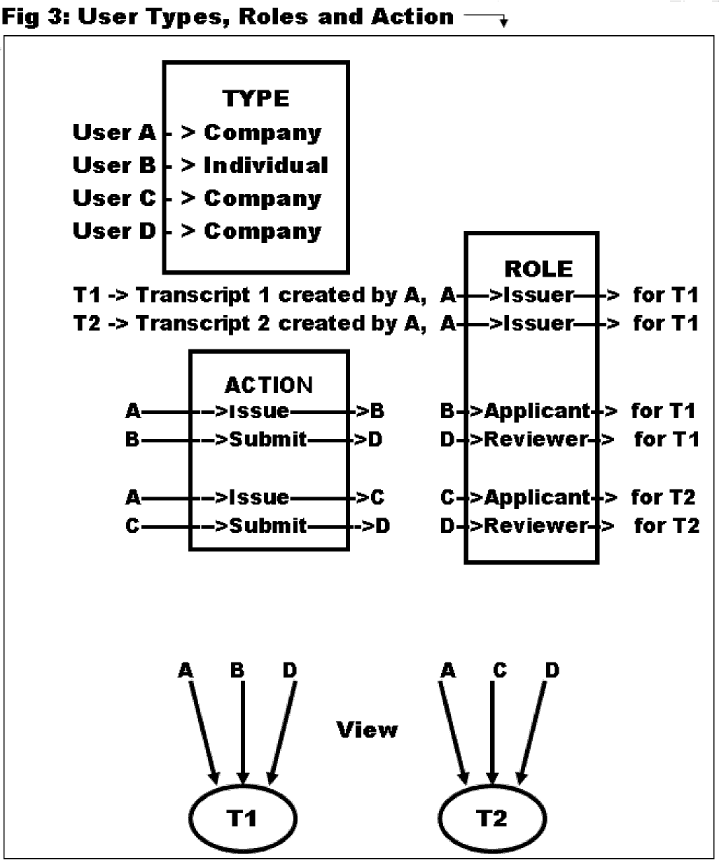

FIG. 3 provides a figurative representation for user types, roles and actions.

Storage on Transcript servers:
registration-table: The registration-table contains an entry for each user and maps the registration information to the OTS-ID and user name/password for the user on the transcript server.

<OTS-ID>-table: A transcript is always linked to a container name. The container name can refer to one or more transcripts. Each user has a <OTS-ID>-table created on the TS that stores container names associated with the user's OTS-ID. This container name is called the Root Transcript Location (RTL).

<RTL>-transfer-table: For every RTL in the system there is a corresponding transfer table called <RTL>-transfer-table. The RTL table contains a list of transcripts, the roles associated with each transcript, a location called a local path for which a client invokes a view request and a location called remote path and remote OTS-ID that will actually serve this view request. If the remote path and remote OTS-IDs self, then the transcripts are actually present as a file in the file system under the local path. This file is the actual transcript file or a link to the actual transcript file that is accessible from the transcript server.

A transcript can be created only by a company user. When a transcript is created, the TS creates an RTL name, which is unique to the transcript server (e.g., timestamp) and assigns the sender OTS-ID for the RTL as self. It also updates the <OTS-ID>-table for the user with this RTL name. This RTL is owned by OTS-ID if the sender is self in the <OTS-ID>-table. Only a owner OTS-ID can create files under the owned RTL. The transfer table for this RTL will contain all local paths to actual locations in the file system and all remote paths and remote OTS-IDs will be self.

When a transcript is issued or submitted the sending transcript server finds the recipient OTS-ID for a recipient unique user identifier from the transcript network server and sends out the envelope for transcript (EFT) information in a Original Transcript Message (OTM) to the transcript network server. This EFT sent out contains the RTL name, RTL description, sender OTS-ID, receiver OTS-ID, local path for each transcript, description for each transcript, and roles associated with each transcript.

The transcript network server finds the appropriate hosting transcript server for the receiver OTS-ID in the OTM and sends this to the recipient transcript server.

The recipient transcript server receives this OTM with the following details. RTL name =<RTL>, sender OTS-ID=<sender OTS-ID>, one or more file path =<RTL>/<file name>.

When this OTM is received the receiving TS creates a new RTL with the name. RTL name =<RTL>@<sender OTS-ID> and updates the <receiver OTS-ID>-table with this RTL name and sender field as <sender OTS-ID> for this RTL. It then proceeds to update the <RTL>@<sender OTS-ID>-transfer-table.

In the transfer table all the file paths got from the OTM go as remote paths and the, <sender OTS-ID> is the remote user The local paths are created from the remote path, by replacing the RTL name portion with the RTL name for this transfer table. In other words <RTL>/<file name> becomes the remote path, <sender OTS-ID> becomes the remote user and <RTL>@<sender OTS-ID>/<file name> becomes local path in <RTL>@<sender OTS-ID>-transfer-table.

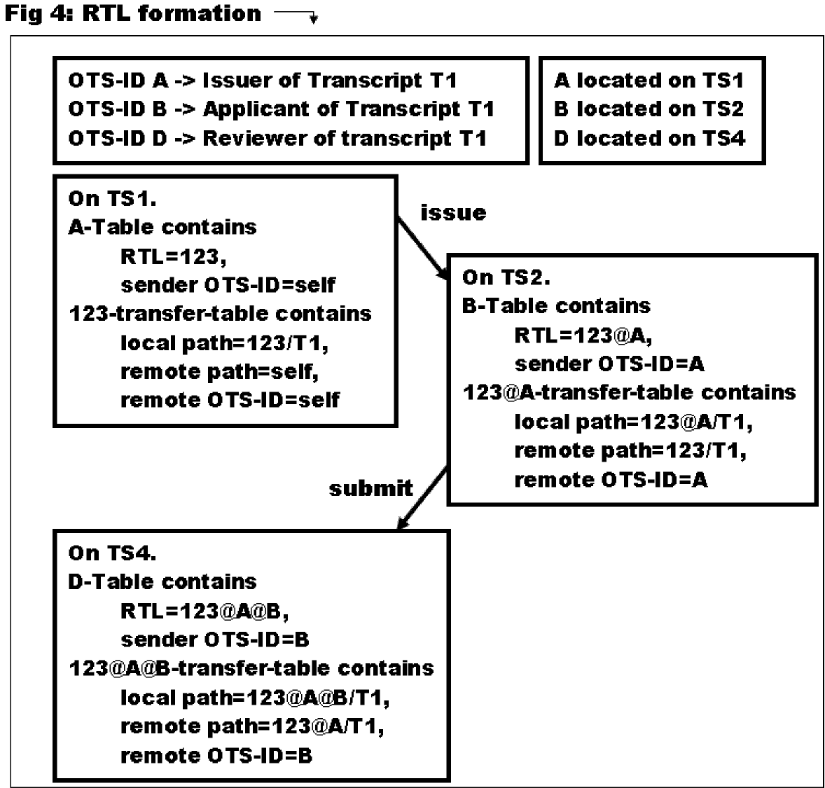

FIG. 4, shows how RTLs form between OTS-IDs during issue, and submit actions.

Storage at Transcript network server:

UUID-OTSID-TS-Table.

This table contains a mapping between unique identifiers, like SSN, the UUID type that identifies if it is a SSN, passport number, etc, and the corresponding OTS-ID. This table also contains the transcript server host name.

Global-Registration-Table.

This table contains the registration information for each OTS-ID. The UUID provided by the user is also a part of the registration information. The UUID-OTSID-table is built out of the Global-Registration-Table.

OTS-Display-Table.

A user during registration can choose to expose some of the users personal information to other users on OTS (e.g. first name and e-mail address). This table contains the mapping between OTS-ID and display information.

TS-Status-Table.

This table maintains the list of active transcript servers. The TNS does not forward OTMs to inactive transcript servers.

Reliable Communication:

The communication between the transcript server and TNS is reliable. When a TS sends an OTM to the TNS, the TNS forwards it to the destination TS. When the destination TS receives this message it acknowledges the receipt to TNS. After a receipt the TNS tells the sending TS that the OTM has successfully reached the destination. If the destination TS is not up or does not acknowledge the successful receipt of a OTM the TNS responds with a failed receipt to the sending TS.

OTM.

Messages between TS that go through the TNS are called OTM (Original Transcript Messages).

EFT OTM: This is the OTM that carries envelope for transcript (EFT) details from sender OTS-ID to a receiver OTS-ID. When the TNS receives an OTM of type EFT it gets the receiver OTS-ID present in the EFT OTM, and sends the EFT OTM to the transcript server hosting the receiver OTS-ID.

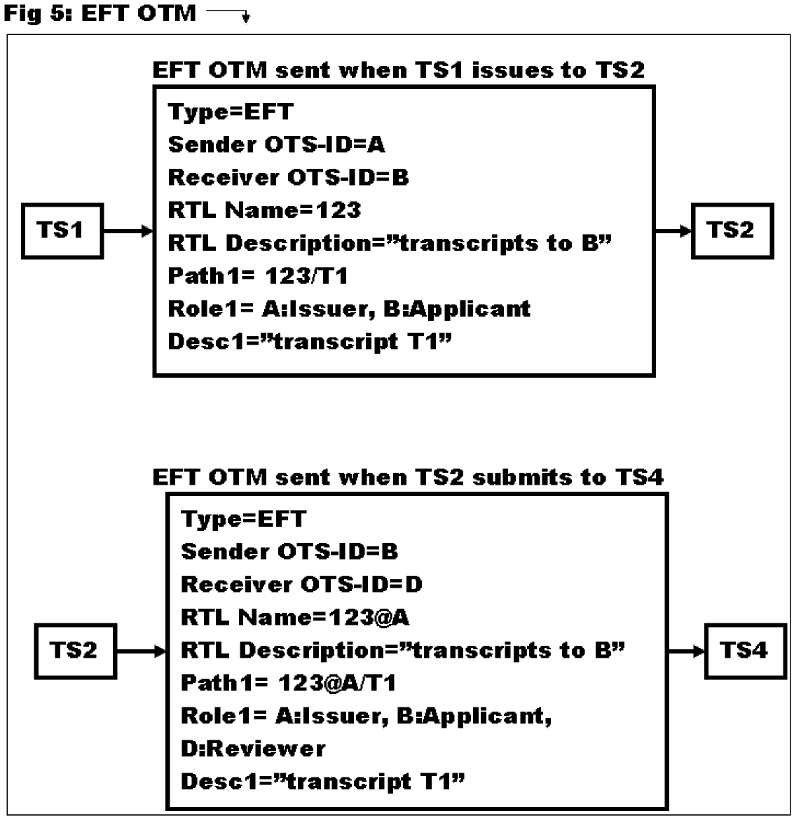

FIG. 5 shows the contents of an EFT OTM for issue and submit actions. Refer FIG. 4 also to get details of users, transcript servers and stored table values.

View OTM: When a view request comes for a local path and if the remote path is not self, the transcript server builds a view OTM that has Interested location (the local path), Interested OTS-ID (the OTS-ID of the user who requested the view), sender OTS-ID (the OTS-ID that sends out this view OTM) and receiver OTS-ID (the OTS-ID mentioned as remote on the transfer-table) and receiver location (the path mentioned as remote on the transfer-table). This view OTM is received by the TNS and forwarded to the TS hosting the receiver OTS-ID.

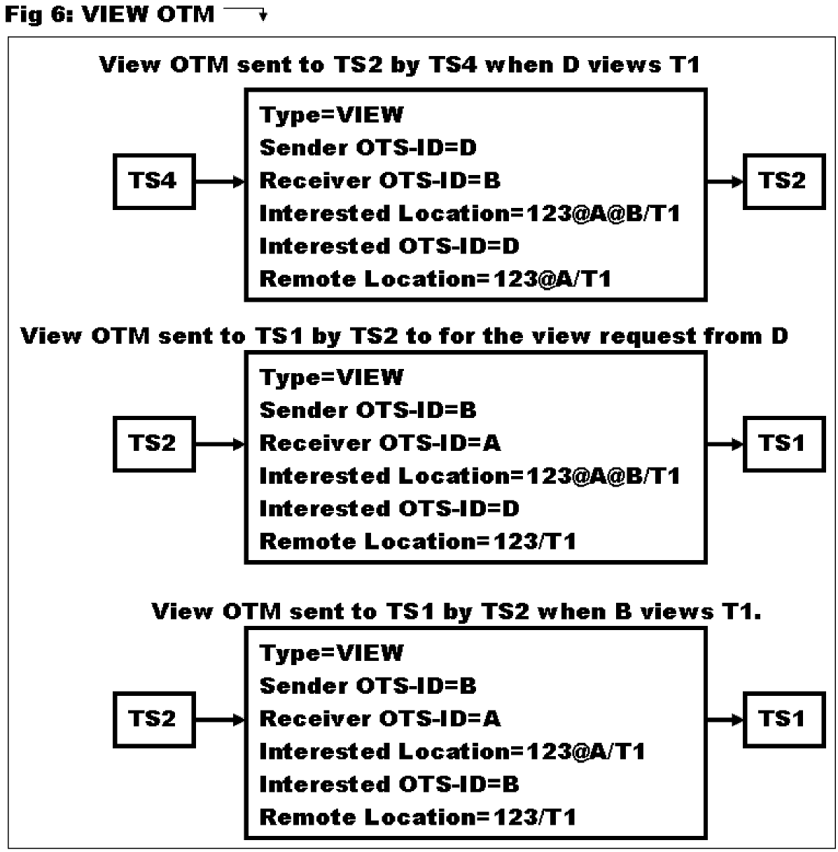

FIG. 6 shows the contents of a View OTM for a view request by the applicant or reviewer. Refer FIG. 4 also to get details of users, transcript servers and stored table values.

Transfer OTM:

The view OTM ends when sent to an issuer. The issuer sends out a transfer OTM which contains the actual transcript, sender OTS-ID=issuer OTS-ID, receiver OTS-ID=interested OTS-ID and interested location on receiver.

The TNS receives this transfer OTM and sends it to the TS hosting the receiver OTS-ID. The TS gets the transcript and gives it to the client waiting at the interested location, to complete the view request.

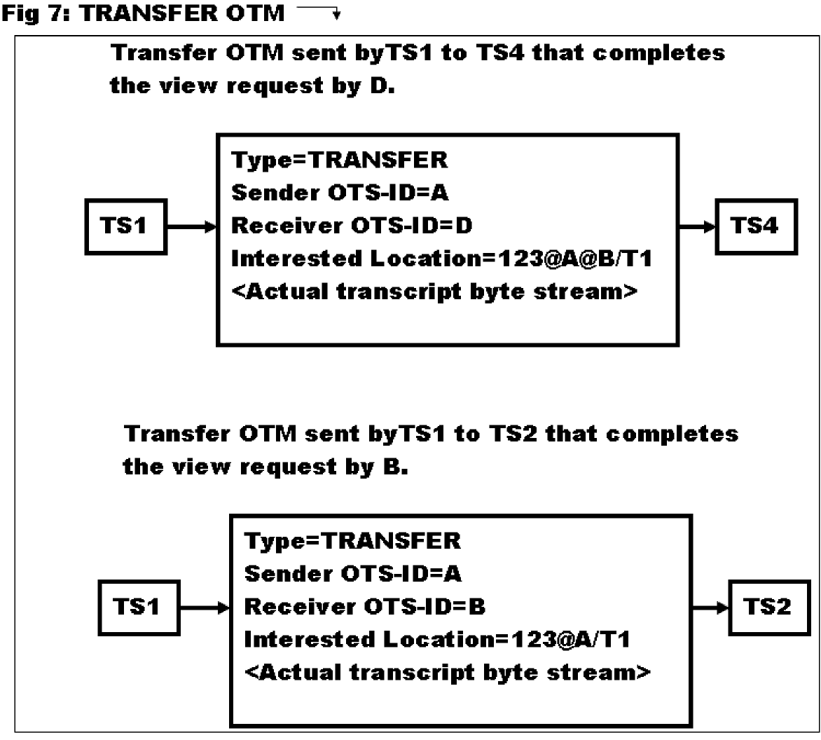

FIG. 7 shows the contents of a transfer OTM sent from issuer to viewer to complete a view request. Refer FIG. 4 also to get details of users, transcript servers and stored table values.

Display OTM:

A TS sends a display OTM to the TNS providing an OTS-ID. The TNS returns with the display name for that OTS-ID that includes personal information classified as display by the user. (e.g., First name and e-mail address).

Look up OTM:

A look up OTM is sent from a TS to the TNS contains the UUID information. The TNS responds back by providing the OTS-ID for the UUID information to the requesting TS.

Identity and Content Security:

VPTN:

The virtual private transcript network is created by assigning public/private keys to each server in OTS. Each transcript server has the public key of the transcript network server. The transcript network server maintains a list of public keys for all transcript servers, on the system.

Any OTM sent by the TS is encrypted with the private key of the transcript server.

When a OTM is received from a transcript server. The TNS finds the originating host name for the message. It then queries its tables and decrypts the message with the public key, got corresponding to the transcript server host name. if the message is not decrypt able it ignores the message. After the message is decrypted it confirms if the sender OTS-ID belongs to the sending transcript server. The TNS then encrypts this message with the private key of the TNS and sends it out to the TS hosting the receiver OTS-ID.

The receiving transcript server, decrypts the message with the TNS public key.

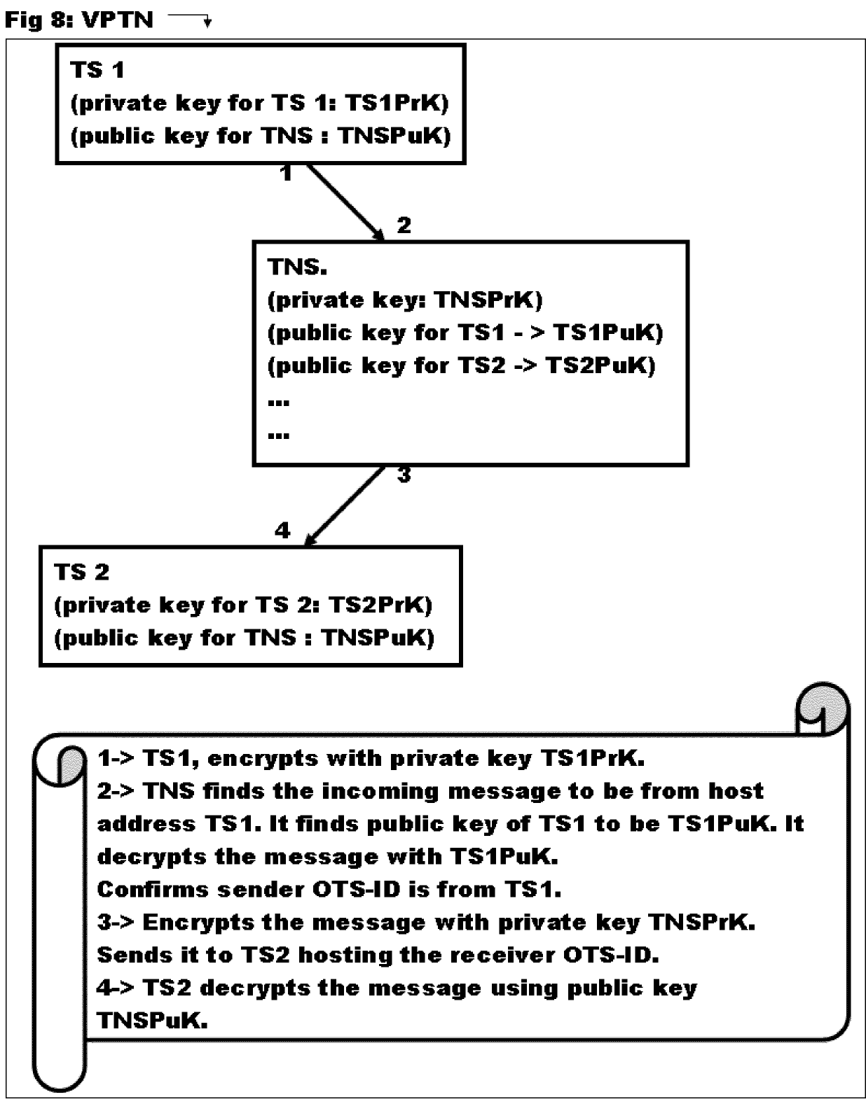

FIG. 8 shows the secured communication between TS and TNS which forms the Virtual private transcript network (VPTN) for OTS.

A company owns a transcript server, and gets the keys from the transcript provider. Individuals are associated with transcript servers owned by Transcript Provider

OTS-ID acts as the address for a user and ties the user to a transcript server and the user's UUID. Hence role identity on a transcript is always guaranteed.

Transcripts are not stored on reviewer or applicant. An issuer's RTL name maps to a location in the file system, that can contain the transcript or a link to the file accessible by the transcript server.

A view OTM from an OTS-ID who is the reviewer, received by another OTS-ID who is the applicant is forwarded to the issuer only if the sender OTS-ID is a reviewer to that transcript. Also an issuer accepts view OTMs only from an applicant of the transcript

A incoming transfer OTM is accepted only if the issuer role on the transcript matches with the sender OTS-ID in the transfer OTM.

An ETM is not forwarded by the TNS if the hosting transcript server does not host the sender OTS-ID in the ETM.

### Operation: Preferred Embodiment

The operation of OTS can be best explained with a real life “like” scenario.

Operation of OTS is not limited to the particular scenario below, but rather can operate wherever there is a need to issue, receive or view original transcripts, by users of OTS.

John Turner is employed with Tecoi Systems, and needs a loan to buy a home.

He applies for a loan to lenders Bank Of Trust and Loan Pal. The lenders need John to submit a verification of employment (VOE) from Tecoi Systems. Tecoi Systems issues a VOE transcript to John “using OTS”. John submits this transcript using to Bank Of Trust and Loan Pal, “using OTS”. Bank Of Trust views John's VOE “using OTS”. Loan Pal views John's VOE “using OTS”. After view of John's VOE, Bank Of Trust finds John's salary insufficient and responds unfavorably to John's request. But Loan Pal finds John's salary to meet their requirements and needs a savings account statement to complete the review of john's loan application. Contra Bank issues John his savings account statement “using OTS”. John submits this transcript to Loan Pal “using OTS”. Loan Pal views John's savings account statement “using OTS”. Loan Pal decides favorably and invites John to sign the necessary papers. After this Loan Pal prepares a transcript and issues it to John “using OTS”. John submits this Loan transcript to IRS as he needs a tax rebate on his loan. IRS views John's Loan transcript “using OTS” and grants the rebate.

All users are registered to their respective transcript servers The transcript servers can talk to each other though the transcript network server.

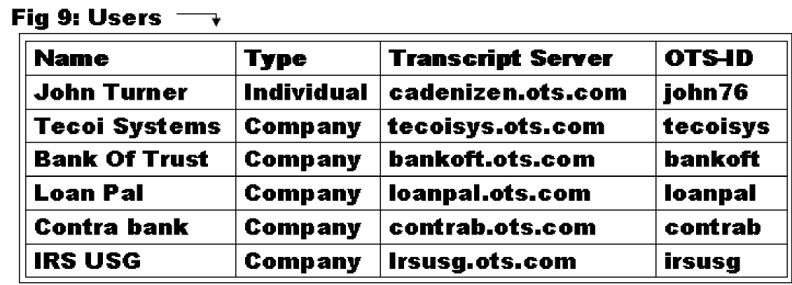

FIG. 9, details the name of each user, the OTS-ID for each user, the transcript server that the OTS-ID is assigned to, and the registration type (Individual or Company) for each user.

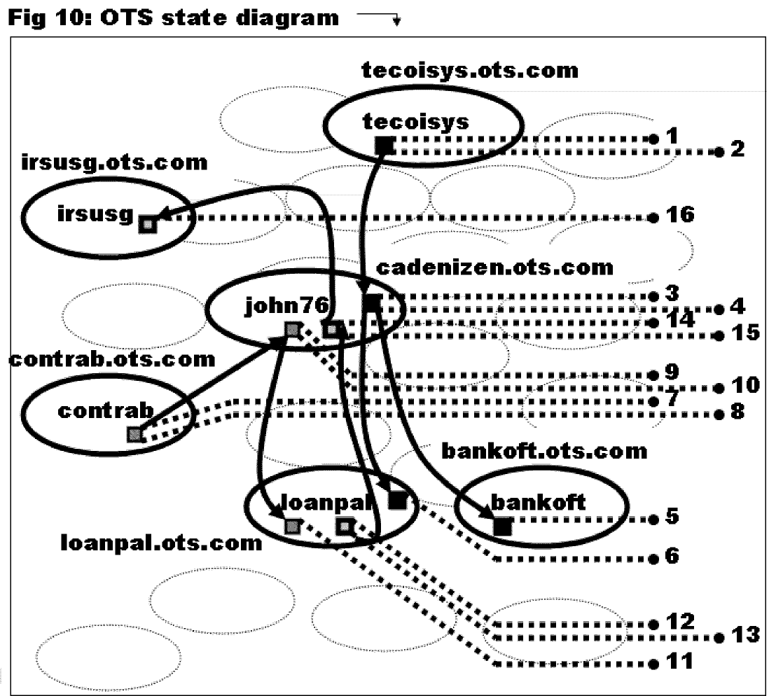

FIG. 10, refers to the OTS state diagram for this particular scenario.

The oval figures are transcript servers. An OTS-ID is mentioned inside the transcript server because that OTS-ID is hosted by the transcript server. The transcript servers involved in this scenario are those oval figures with a continuous line. The transcript servers not involved in this scenario are those oval figures with a dashed line. The figure shows three types of small squares. This means that there are three separate transcript chains involved. (VOE, Account Statement and Loan). The top of the square refers to the state when a transcript is received. The bottom of the square refers to the state when a transcript is sent. These states are also numbered in sequence with dotted lines pointing to the top and bottom of the small squares.

Tecoi Systems has prepared the PDF VOE.pdf for John.

Tecoi Systems logs into transcript server (TS):
tecoisys.ots.com and is recognized by its TS as OTS-ID tecoisys. tecoisys enters as issuer. tecoisys creates the VOE.pdf on tecoisys.ots.com. This file can be created by transferring the actual transcript to the TS or by creating it as a link on the TS that points to the actual file located in another computer system and accessible from tecoisys.ots.com. tecoisys chooses to transfer the actual file.

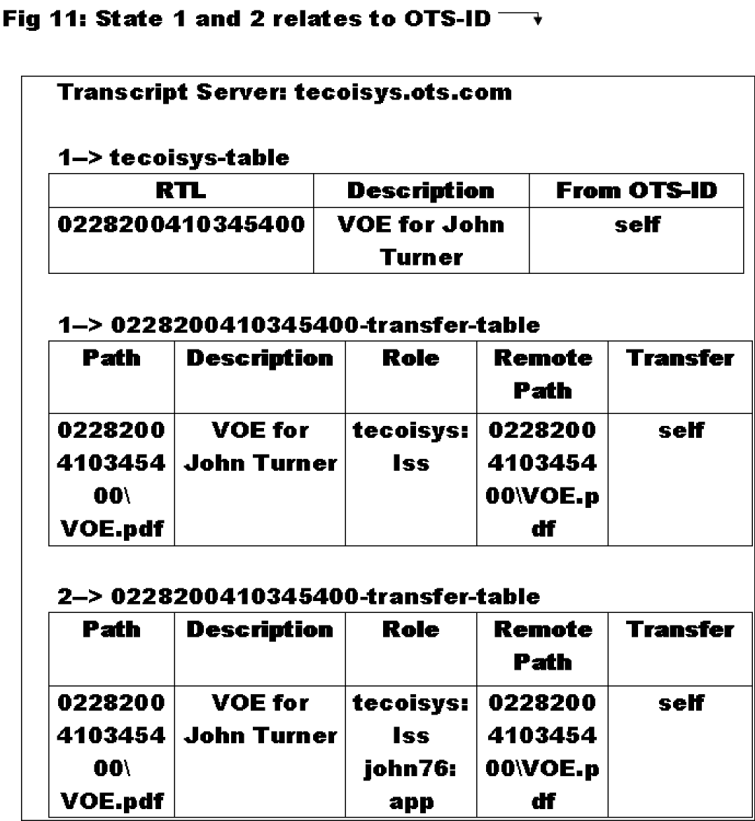

State 1: Refer to FIG. 11: When transcript server:
tecoisys.ots.com receives a transcript from tecoisys, it creates an RTL 0228200410345400, which is a directory and stores VOE.pdf under this RTL. It then proceeds to update tecoisys-table and 0228200410345400-transfer-table.

State 2: Refer to FIG. 11: Once the transfer is complete tecoisys can now issue VOE.pdf to a UUID. tecoisys inputs john's SSN, which it already has in its employee records and issues the transcript. tecoisys updates the description for that RTL and description for the file under the RTL as “VOE for John Turner”. then ‘issues’ the transcript VOE.pdf to the UUID for John. A look up OTM is sent by tecoisys.ots.com for the UUID to the TNS. The TNS responds by providing OTS-ID as john76. tecoisys.ots.com updates the 0228200410345400-transfer-table with role information as tecoisys:issuer, john76:applicant. It constructs an EFT OTM and sends it out to the TNS. The TNS receives this EFT OTM, and forwards it to the TS hosting john76.

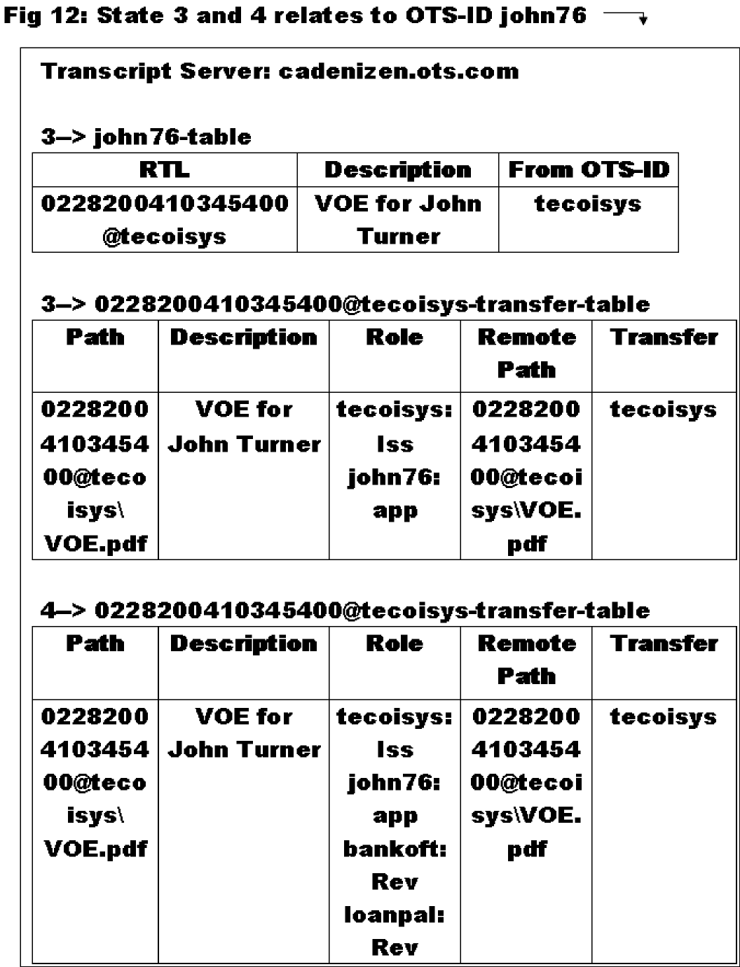

State 3: Refer to FIG. 12: John Turner logs into transcript server:cadenizen.ots.com and is recognized as OTS-ID john76. He can only enter as an applicant, because his registration is of Individual status. He sees that a RTL has arrived from tecoisys with description “VOE for John Turner”, from sender tecoisys. He clicks that RTL to see more information, and finds a link that says VOE.pdf, description “VOE for John Turner”, role information says tecoisys:issuer, john76:applicant.

When John clicks on this link, view OTMs are generated that reach tecoisys.ots.com. tecoisys.ots.com responds with a transfer OTM that is received by cadenizen.ots.com. John's client application waits on the path for VOE.PDF. This local path also matches the Interested path in the received transfer OTM. cadenizen.ots.com gives the transcript to the client waiting on the local path, to complete the view request.

State 4: Refer to FIG. 12: John ‘submits’ the transcript VOE.PDF to lenders with UUIDs, www.bankoftrust.com and www.loanpallenders.com by clicking on the submit button next for VOE.PDF.

cadenizen.ots.com sends out look up requests to TNS for both the UUIDs. When the OTS-IDs are received it updates the RTL-transfer-table with role information as tecoisys:issuer, john76:applicant, bankoft:reviewer, loanpal:reviewer. cadenizen.ots.com sends EFT OTMs to receiver OTS-IDs loanpal and bankoft, which are received by the TNS and forwarded to their respective transcript servers.

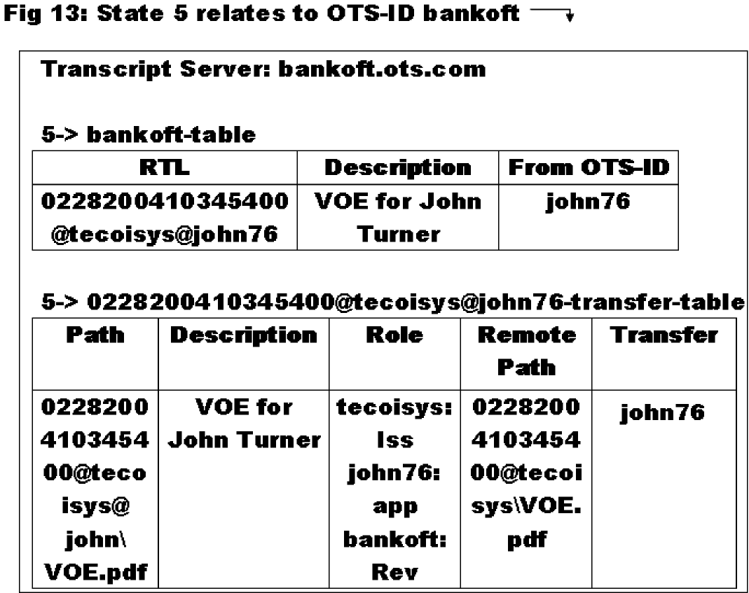

State 5: Refer to FIG. 13: Bank Of Trust logs in to transcript server bankoft.ots.com and is recognized as OTS-ID bankoft by its transcript server. bankoft enters as reviewer. It has received a RTL from john76 for which bankoft is the reviewer. bankoft clicks on the RTL and sees a link for file VOE.PDF. bankoft views the transcript by clicking on the link which generates view OTMs received by cadenizen.ots.com. cadenizen.ots.com in turn finds the issuer OTS-ID and location (remote OTS-ID and remote location) and sends an view OTM which is received by tecoisys.ots.com. tecoisys.ots.com responds with a transfer OTM that is received by bankoft.ots.com. Bankoft's client application waits on the local path for VOE.PDF on bakoft.ots.com. This local path also matches the Interested path in the received transfer OTM. bankoft.ots.com gives the transcript to the client waiting on the local path, to complete the view request.

Bank of trust does not find John's salary to satisfy their minimum requirements for a loan. Bank of trust telephones, e-mails or snail mails John denying him the loan.

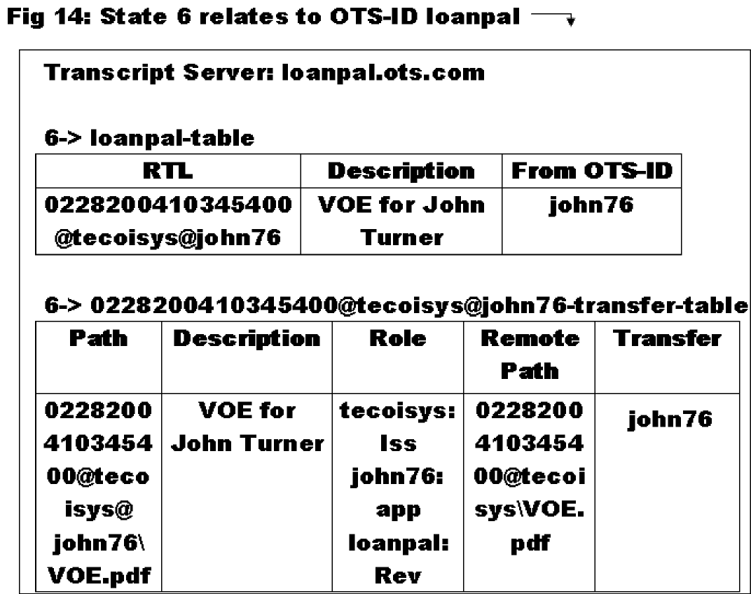

State 6: Refer to FIG. 14: Loan Pal logs in to transcript server loanpal.ots.com and is recognized as OTS-ID loanpal by its transcript server. loanpal enters as reviewer. It has received a RTL from john76 for which loanpal is the reviewer. loanpal clicks on the RTL and sees a link for file VOE.PDF. loanpal views the transcript by clicking on the link which generates view OTMs received by cadenizen.ots.com. cadenizen.ots.com in turn finds the issuer OTS-ID and location (remote OTS-ID and remote location) and sends an view OTM which is received by tecoisys.ots.com. tecoisys.ots.com responds with a transfer OTM that is received by loanpal.ots.com. loanpal's client application waits on the local path for VOE.PDF on loanpal.ots.com. This local path also matches the Interested path in the received transfer OTM. loanpal.ots.com gives the transcript to the client waiting on the local path, to complete the view request.

Loan pal finds John's salary to meet their minimum requirements for a loan. Loan Pal telephones john, e-mails or snail mails him and requests John provide them a savings account bank statement.

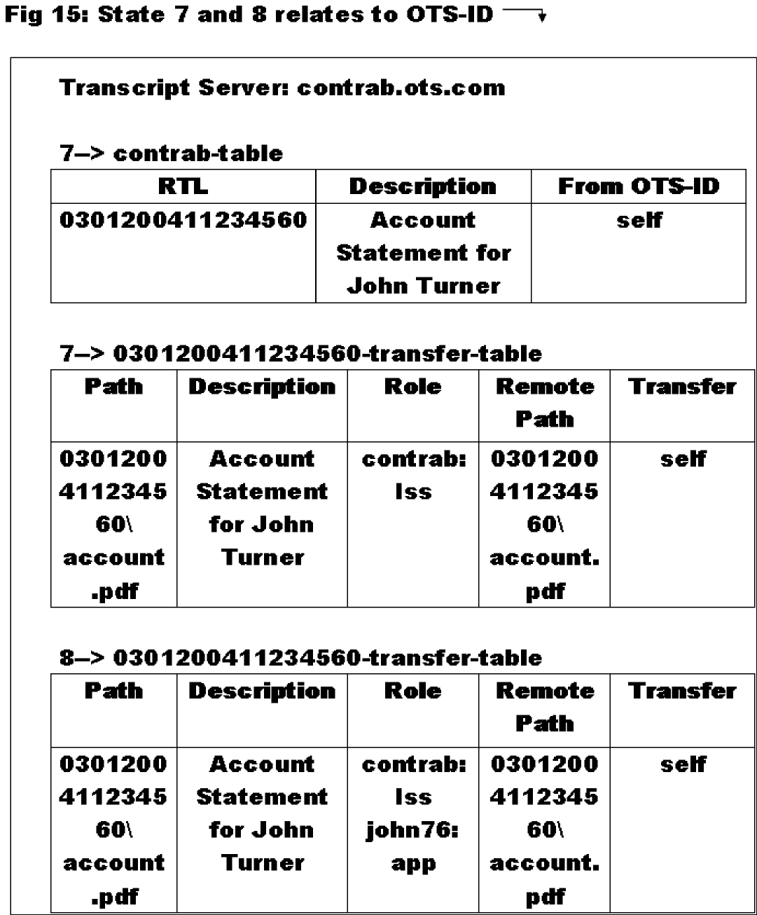

State 7: Refer to FIG. 15: Contra bank logs into transcript server contrab.ots.com and is recognized as OTS-ID contrab. contrab enters as issuer and creates a file Account.PDF and transfers it to contrab.ots.com. When the TS receives this file it creates an RTL 0301200411234560 and assigns this file under the RTL. The transcript table is updated with role information as contrab:issuer

State 8: Refer to FIG. 15: After this contrab proceeds to ‘issue’ this transcript to John's UUID. contrab.ots.com requests the OTS-ID for John's UUID. When this OTS-ID is received contrab updates the transfer table. The role information in the transfer table reads contrab:issuer, john76:applicant. contrab.ots.com creates a EFT OTM and sends it out to the TNS.

The TNS receives this OTM and forwards it to cadenizen.ots.com for OTS-ID john76.

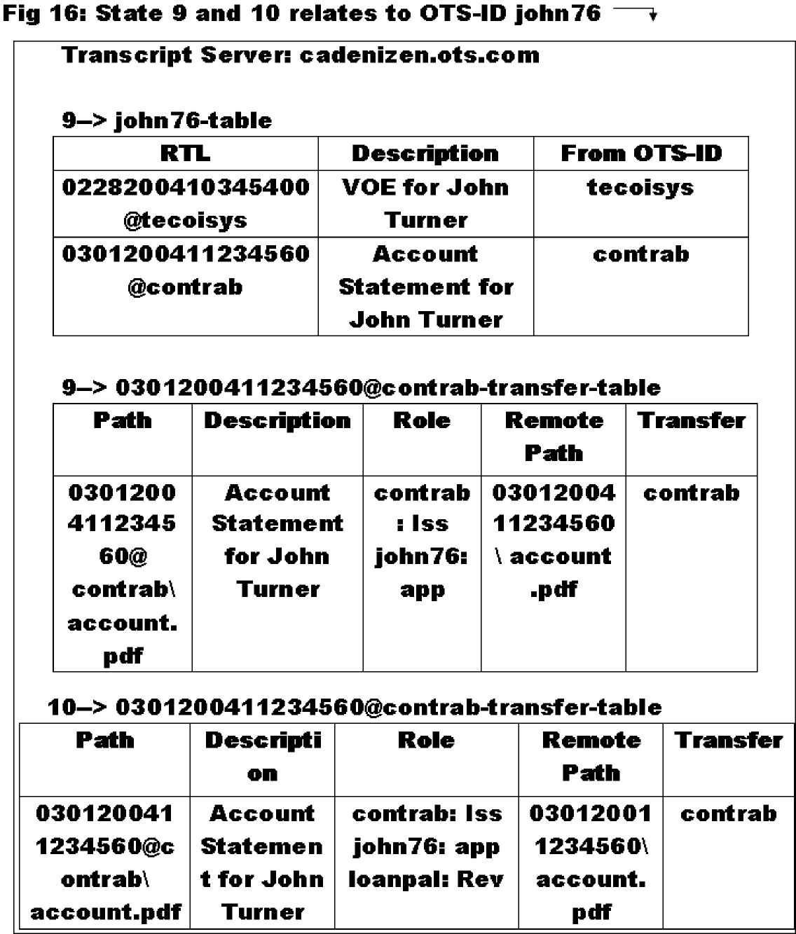

State 9: Refer to FIG. 16: At cadenizen.ots.com john76 now sees two RTLs. The previously received RTL from tecoisys and the newly arrived RTL from contrab. The transfer table has roles for file Account.PDF as contrab:issuer, john76:applicant.

John views this transcript. This causes view OTMs to go to contrab.ots.com and contrab.ots.com replies with the transcript for john76's local path by sending a transfer OTM.

State 10: Refer to FIG. 16: John76 now chooses the Ac-count.PDF link under this RTL and submits it to www.loanpallenders.com. cadenizen.ots.com gets the OTS-ID for the website from TNS and updates the transfer table with role information, contrab:issuer, john76:applicant,loanpal:reviewer. It creates an EFT OTM for OTS-ID loanpal and sends it to the TNS, which forwards this OTM to TS loanpal.ots.com for OTS-ID loanpal.

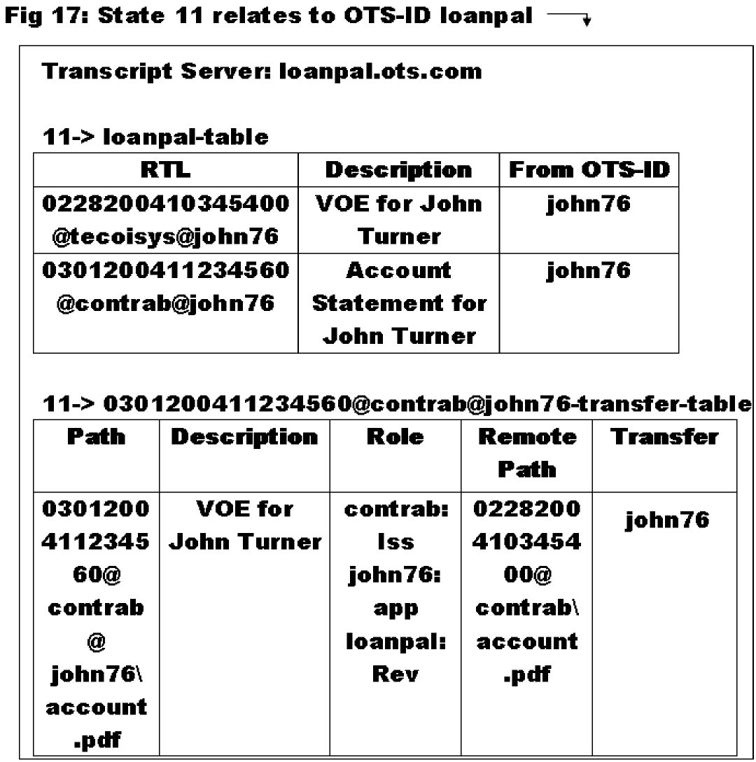

State 11: Refer to FIG. 17: loanpal enters as reviewer. There are two RTLs from OTS-ID john76. One that has description, VOE for John Turner and the other that has description “Account Statement For John Turner”. loanpal clicks the RTL for account statement and sees the Account.PDF link in it. When loanpal clicks this link view OTMs are sent from loanpal.ots.com bound for OTS-ID john76 and a localpath on cadenizen.ots.com. But since this local path in turn refers to the issuer path as remote, cadenizen.ots.com sends out a view OTM to contrab.ots.com and OTS-ID contrab. Contrab.ots.com receives the interested local path, finds that the remote is self and sends a transfer OTM to loanpal and the local path for account.pdf on loanpal.

Loanpal has all the information forjohn76's loan. He invites john to complete the formalities and enters as issuer and transfers file Loan.PDF, to loanpal.ots.com. This is the transcript of loan details for john turner from Loan pal.

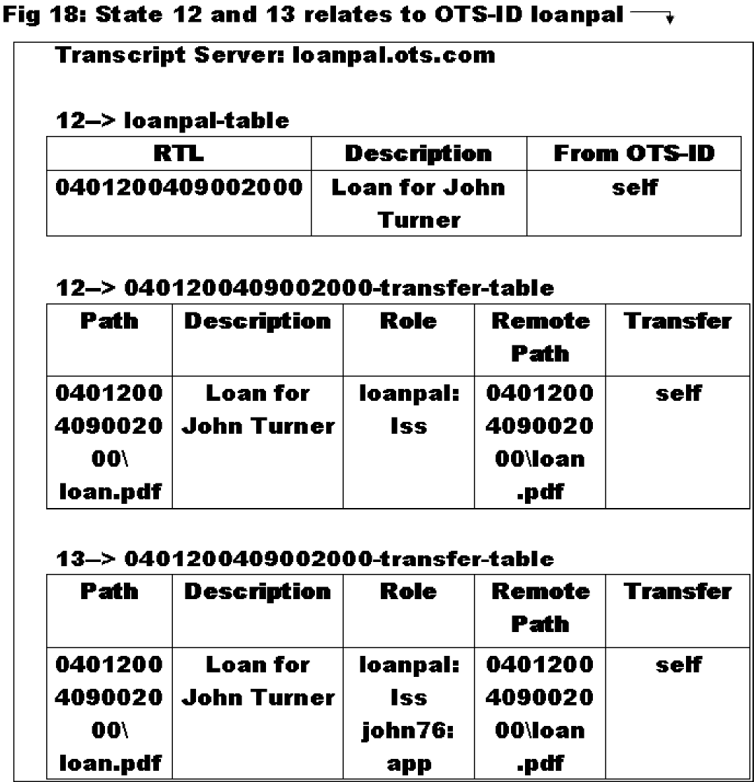

State 12: Refer to FIG. 18: loanpal.ots.com creates a new RTL 0401200409002000 and assigns file Loan.PDF under this RTL. The transfer table has roles for this file as loanpal:issuer.

State 13: Refer to FIG. 18: loanpal proceeds to issue this file to John's UUID. loanpal.ots.com updates the transfer table with the OTS-ID for john got from TNS. The roles for Loan.PDF is updated as loanpal:issuer, john76:applicant. loanpal.ots.com creates an EFT OTM and sends it to the TNS, bound for OTS-ID john76.

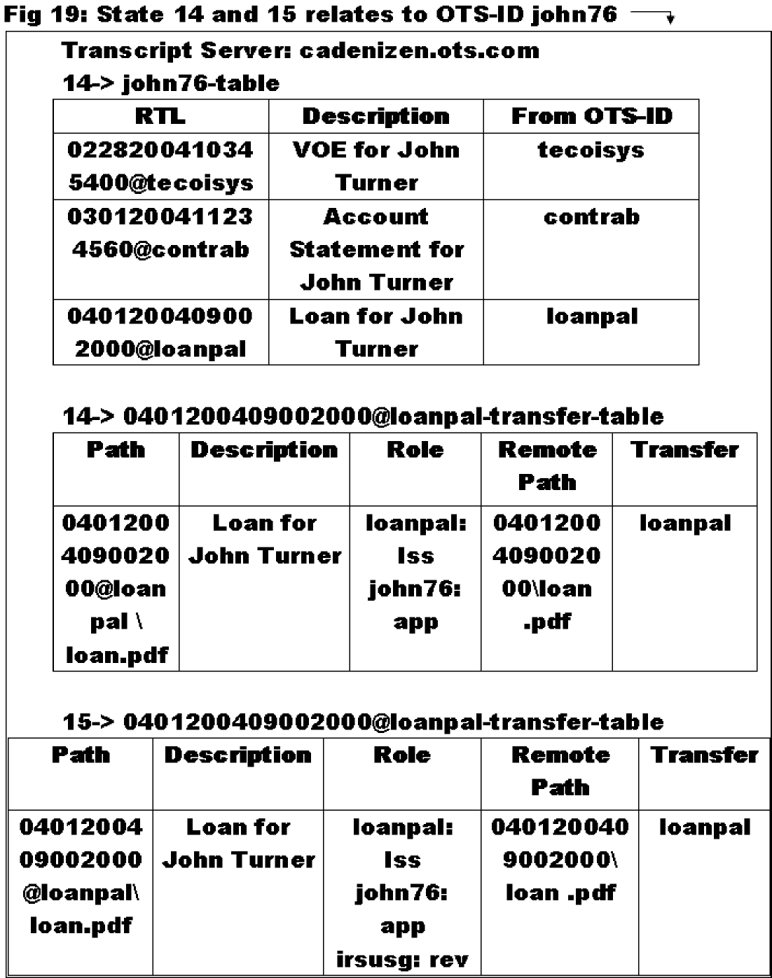

State 14: Refer to FIG. 19: john76 receives another RTL. This RTL is from loanpal. It contains a link to the Loan.PDF issued by loanpal. The roles for Loan.PDF is issuer:loanpal, applicant:john76

State 15: Refer to FIG. 19: john76 views this transcript and ‘submits’ it to www.irsusg.com for a tax rebate. cadenizen.ots.com talks to TNS and gets the reviewer OTS-ID as irsusg. It updates the role information for Loan.PDF in transfer table for this RTL as issuer:loanpal, applicant:john76, reviewer:irsusg. cadenizen.ots.com creates a EFT OTM and sends it to the TNS which forwards the OTM to the TS hosting the reviewer OTS-ID irsusg.

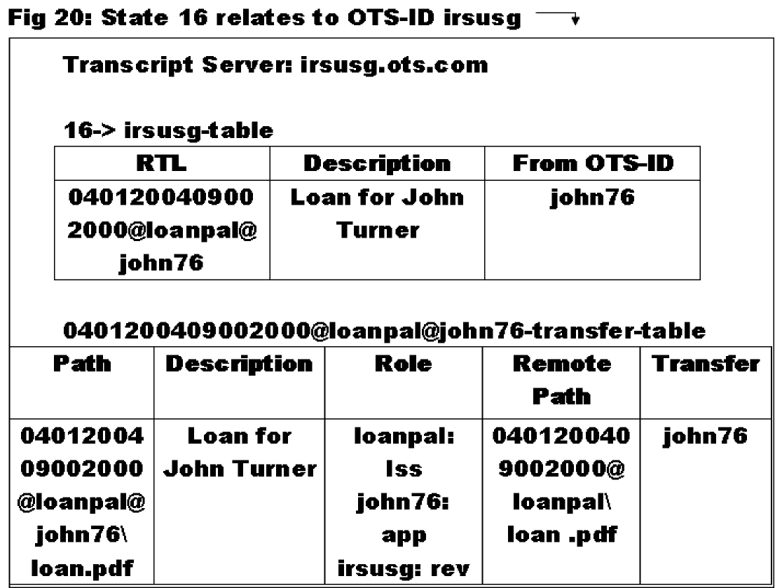

State 16: Refer to FIG. 20: IRS logs in to irsusg.ots.com and is recognized as OTS-ID irsusg. It has received a RTL from john76 with description “Loan for john Turner”. irsusg views this document, which causes irsusg.ots.com to send view OTMs to OTS-ID john76, cadenizen.ots.com to send view OTMs for OTS-ID loanpal, and loanpal.ots.com sends a transfer OTM to OTS-ID irsusg that contains the transcript.

This example illustrates how transcripts are got directly from the issuer using OTS. Applicants or reviewers need not store their originals any more. They can directly get it on demand from the issuer. Issuer has complete control and can stop access to an original transcript any time by just deleting that transcript or taking the transcript server out of the virtual private transcript network.

### SUMMARY

The features addressed by OTS in order to provide effective original transcript service are: Availability, Scalability, Manageability and Security.

Availability: For a transcript to be viewed the transcript servers hosting the relevant OTS-IDs must be up and be able to talk to the transcript network server.

If a user needs to submit a transcript the user can go to a computer connected to the Internet, and log in to the user's transcript server and submit an issued transcript to the reviewer. The reviewer receives it and views it directly from the issuer.

Issuers have absolute control of a transcript. They can delete it or replace it.

A view request to a transcript will fail it was deleted by the issuer. A view request to a transcript will fetch the latest version if it was replaced by the issuer.

An issuer deletes a transcript when it is no longer valid. An example of this could be a need by an employer to delete verification of employment transcript since the employee is no longer employed by the employer.

An issuer can also replace the document that is invalid with a document that is valid. In such cases the document need not be issued again. A view request by the applicant or reviewer will pick this new transcript. An example of this could be a driving license transcript that is replaced by DMV after its expiry.

Since an applicant does not store original transcripts at applicant's end, an applicant may still want the previous version of the transcript or the deleted transcript. In these cases the applicant can request the issuer not to delete a transcript or issue a newer version as a new RTL. Alternately he can use the services of a notary who is registered on OTS as a company.

In this option, the applicant can submit the transcript to the notary company for review. The notary views the transcript from the issuer. The notary can then act as issuer and issue this transcript back to the applicant.

Scalability:

A large company has many employees. An existing large company would already maintain transcripts for employees at various locations and means.

The advantage of OTS is transcript servers owned by the companies can be used to issue transcripts already maintained by the company without actually having to store them on the transcript server. The companies can also store them if they chose to maintain the transcript on the transcript server. This is because the location of a transcript at the issuer is the actual file or a link to another destination that is accessible from the transcript server. So this link could point to another file in an internal computer maintained by the company.

When a company finds one transcript server insufficient due to growth in database table content, then multiple transcript servers can work in a proxy configuration. In these cases the proxy transcript server acts as the transcript server for the OTS-ID which hides other transcript servers behind it. The hidden transcript servers can only talk to the proxy, and are not known to TNS. The proxy is configured to route RTLs based on the timestamp, in the RTL name, to the different transcript servers it hides. This distributes the RTL storage across transcript servers owned by the company and still maintain a single identity for a company on OTS.

A transcript network server contains information about all OTS-IDs. So there may be a need for the transcript provider to maintain more than one transcript network server and distribute the load of transcript servers between these transcript network servers. With this configuration a TS will always send an OTM to its assigned TNS. If the destination is not known the TNS forwards it to other known TNS servers who could know about the destination OTS-ID. The OTS-IDs maintained with one TNS should not clash with the OTS-IDs maintained in another TNS, when more than one TNS belongs to the system.

Manageability:

The main advantage of OTS applicants and reviewers need not store original transcripts, at their end. Issuers can use OTS to issue transcripts, and have full control of the transcript. OTS also provides identity information for a transcript of all involved parties. This enables an user to search for transcripts based on criteria which include issuer, applicant, reviewer, description, and file names. An issuer can readily know to who all a transcript was issued to. An applicant can readily know from whom a transcript was issued and to who all that transcript was submitted to. A reviewer will readily know from whom a transcript was received and who the issuer for the transcript is.

Issuing, receiving, submitting or viewing a original transcript is achieved simply by logging to the user's transcript server. A user even while at a remote location and if needed to submit a transcript for review, can log in to the user's transcript server through a web browser and submit it for review.

With OTS users can eliminate e-mailing or snail mailing transcripts between an issuer, applicant and reviewer.

Security:

OTS generates the OTS-ID which is unique for a user and ties that user to a transcript server. The OTS-ID is generated after a background check done by the transcript provider for the user, based on the registration information.

This OTS-ID is mapped to the UUID information provided by the user during registration.

When a transcript is issued or submitted, it is always sent to a UUID by the user. For a company this UUID can be a company web site name. For an individual the UUID can be a SSN number or passport number.

Companies own their own transcript servers. Hence they have complete control of the transcripts issued by them.

A receiver of a transcript who is an applicant or reviewer does not store the transcript. When a transcript is viewed by the applicant or reviewer the transcript always comes from the issuer. When a transcript is viewed by an user, a view OTM gets sent to the remote OTS-ID for a remote path. The issuer completes the view request only if the request comes from an applicant of that transcript. An applicant forwards a view request to the issuer only if the request comes from a reviewer of that transcript.

The transcript provider provides public/private keys for each server. The TNS maintains a list of public keys of all transcript servers it serves. Each transcript server maintains the public key of the TNS it talks to. When a message is sent from a transcript server it is encrypted using the transcript server's private key. The TNS finds the source host name and gets the matching public key and tries to decrypt the message. It then confirms the sender OTS-ID in the message is from the same transcript server it received the message from.

The TNS then encrypts the message with its private key and sends it to the transcript server hosting the destination OTS-ID. The receiving transcript server decrypts the message with the TNS public key.

By following these steps OTS achieves identity security and content security for a transcript.

1. a method and system to securely issue submit and view original electronic transcripts by hosting users, who act in one of the roles of issuers applicants or reviewers for a transcript, on servers called transcript servers that communicate with each other through the transcript network server, where a transcript server can host one or more users, where a issue refers to sending envelope for transcript information, from issuer to applicant and submit refers to sending envelope for transcript information from applicant to reviewer, and view refers to sending the actual transcript from the issuer to applicant or issuer to reviewer:

2. A method and system to securely issue submit and view original electronic transcripts as recited in claim 1 where transcript servers store envelope for transcript information that includes the transcript path, by creating the path on the issuer as a link to self where this path on the transcript server contains a file or link to a file on another computer system accessible by the transcript server, by creating the path on the applicant as a link to path on issuer, by creating path on reviewer as a link to path on applicant.

3. A method and system to securely issue submit and view original electronic transcripts where identity information, of users is guaranteed, by the transcript server communicating with the transcript network server during registration of the user and the transcript network server generating an address for each user on the system called the OTS-ID, which is unique in the system and also ties a user to the transcript server and user's registration information which contain one or more unique identifiers for the user, by storing all OTS-IDs present in the system, their respective hosting servers and registration information, on the transcript network server.

4. A method and system to securely issue submit and view original electronic transcripts as recited in claim 1 by identifying a user as company or individual and assigning possible roles of issuer, applicant and reviewer on a transcript for a company user, and possible roles of applicant on a transcript for an individual user, where a company is a user who register with company specific information like EIN and an individual is a user who registers with individual specific information like SSN.

5. A method and system to securely issue submit and view original electronic transcripts as recited in claim 1 where a company who creates a transcript becomes the issuer for the transcript and can issue it another company or individual which becomes the applicant, where an applicant can receive the issued transcript to it and submit the transcript to a company other than the issuer, and the submitted to company becomes the reviewer.

6. A method and system to securely issue submit and view original electronic transcripts as recited in claim 1 to an applicant by issuer issuing the transcript to a unique ID like SSN, or company web site name, by the issuer's transcript server translating the unique ID to an OTS-ID by communicating with the transcript network server, by the issuer's transcript server sending the envelope for transcript information bound for the OTS-ID to the transcript network server, by the transcript network server finding the hosting transcript server for the OTS-ID and sending the envelope for transcript information to the hosting transcript server.

7. A method and system to securely issue submit and view original electronic transcripts as recited in claim 1 to a reviewer by the applicant submitting the transcript to a unique ID like company web site name, by the applicant's transcript server translating the unique ID to an OTS-ID by communicating with the transcript network server, by the applicant's transcript server sending the envelope for transcript information bound for the OTS-ID to the transcript network server, by the transcript network server finding the hosting transcript server for the OTS-ID and sending the envelope for transcript information to the hosting transcript server.

8. A method and system to securely issue submit and view original electronic transcripts as recited in claim 1 where a view request by an applicant, causes the applicant's transcript server to send a view request for local applicant path and remote issuer path to the transcript network server and the transcript network server forwards the request to the transcript server of the issuer. The issuer transcript server sends the transcript from the local issuer path to the transcript network server bound for the local applicant path, and the transcript network server forwards this transcript to the applicant's transcript server which the applicant's transcript server receives and matches it to the local path on the applicant's server.

9. A method and system to securely issue submit and view original electronic transcripts as recited in claim 1 where a view request by a reviewer, causes the reviewer's transcript server to send a view request for local reviewer path and remote applicant path to the transcript network server and the transcript network server forwards the request to the transcript server of the applicant. The applicant transcript server receives this request and forwards it to the network server for local reviewer path and remote issuer path and the transcript network server forwards the request to the transcript server of the issuer. The issuer transcript server sends the transcript from the local issuer path to the transcript network server bound for the local reviewer path, and the transcript network server forwards this transcript to the reviewer's transcript server which the reviewer's transcript server receives and matches it to the local path on the reviewer's server.

10. A method and system to securely issue submit and view original electronic transcripts as recited in claim 1 where transcripts are always associated with a container name called root transcript location (RTL), where RTL at the issuer is a directory location in addition to a name in the database table that contains the transcript as a file or link to a file in another computer system accessible by the hosting transcript server, where issuer can create one or more transcripts under the RTL, and thus make these transcripts related, where a RTL name at the sender is received by the destination as part of the envelope for transcript information, where a RTL is named at the destination by joining the RTL name at the sender with the symbol ‘@’ and the sender OTS-ID, where local transcript path always contain a container name and file name, where the container name is the RTL name.

11. A method and system to securely issue submit and view original electronic transcripts as recited in claim 1 where notarization of a transcript is performed by a notary agent registered as a company in OTS who issues a transcript to the applicant which is a notarized version of a transcript issued by another company to the applicant, which the applicant had submitted for review to the notary agent company.

12. A method to securely issue, submit and view transcripts as recited in claim 1 where a transcript server is assigned a private key that it uses to encrypt out going messages, where the transcript server knows the public key of the transcript network server that it uses to decrypt incoming messages, where the transcript network server knows the public keys of all transcript servers that communicate to it, where the transcript network server finds the host transcript server address of an incoming message and retrieves the corresponding public key from its table and decrypts the message with that public key, where after successful decryption the transcript network server confirms the sender OTS-ID belongs to transcript server which sent the message, where the transcript network server encrypts the message with its private key and sends it to the transcript server hosting the receiving OTS-ID.
